(19) United States
(12) Patent Application Publication
(10) Pub. No.: US 2021/0205325 A1
(43) Pub. Date: Jul. 8, 2021

(54) METHODS OF TREATING A SUBJECT HAVING AN INFECTIOUS DISEASE

(71) Applicant: INMEDIX, INC., Normandy Park, WA (US)

(72) Inventor: Andrew James Holman, Seattle, WA (US)

(21) Appl. No.: 17/210,899

(22) Filed: Mar. 24, 2021

Related U.S. Application Data
(60) Provisional application No. 62/994,419, filed on Mar. 25, 2020.

(51) Int. Cl. A61K 31/5513; A61K 31/426; A61K 31/4196; A61K 45/06

(52) U.S. Cl. CPC A61K 31/5513; A61K 45/06; A61K 31/4196; A61K 31/426

(57) ABSTRACT

Provided herein are methods of treating a subject having an infectious disease, comprising co-administering to a subject having an infectious disease: an effective amount of an ANS optimizing therapy; and a treatment for the infection disease, wherein co-administering the ANS optimizing therapy improves a prognosis for treatment of the infectious disease.

### REFERENCE TO RELATED APPLICATIONS

This application claims priority to U.S. Provisional Application No. 62/994419, filed Mar. 25, 2020, the entirety of which is incorporated herein by reference.

### BACKGROUND

### Field

Methods and compositions in the field of medicine and infectious diseases are disclosed.

### Description of the Related Art

The immune system reacts to foreign threats, e.g. pathogens, cancer and foreign bodies. Its response can be gauged in two ways: sensitivity and virulence.

Sensitivity can have gradations due to the nature of the threat. Some pathogens can elicit an immediate response, while some do not. This may be to allow the body to respond to pathogens by generating immunologic alarm commensurate with their threat. Evolutionarily, slow immune responses can be detrimental. If a subject's immune system inappropriately responds slowly when intensive immune mobilization is desired, death may result. Survivors may tend to have greater inclination to respond quickly to that specific pathogen and propagate more.

Virulence is the intensity of immune activity—how aggressively the response is executed rather than how quickly the threat is appreciated.

Both components can be impacted by the brain through the autonomic nervous system (ANS), which in turn can be divided into the sympathetic (fight-or-flight) and parasympathetic (rest/restorative) components. Both components can be continuously active and competing for predominance depending on bodily needs—resting or fleeing. The former can act primarily through epinephrine and norepinephrine, while the latter can be managed primarily by acetylcholine.

### SUMMARY

Provided herein is a method of treating a subject having an infectious disease, comprising co-administering to a subject having an infectious disease: a therapeutically effective amount of an ANS optimizing therapy; and a treatment for the infectious disease, wherein co-administering the ANS optimizing therapy improves the prognosis for treatment of the infectious disease. In some embodiments, the ANS optimizing therapy and the treatment are administered concurrently. In some embodiments, the ANS optimizing therapy and the treatment are administered simultaneously. In some embodiments, the ANS optimizing therapy is administered before administering the treatment. In some embodiments, the ANS optimizing therapy is administered after administering the treatment.

In some embodiments, the method includes testing the subject for the infectious disease before co-administering. Optionally, testing is performed before co-administering.

In some embodiments, the ANS optimizing therapy comprises one or more of VNS, and/or an ANS optimizing agent. In some embodiments the ANS optimizing agent comprises an antidepressant, an anxiolytic, an anticonvulsant, a D₃ agonist or antagonist, a nicotinic acetylcholine receptor agonist or antagonist, an alpha-7 nicotinic receptor agonist or antagonist, a GABA_{A} receptor agonist or antagonist, an alpha-1 receptor agonist or antagonist, and an alpha-2 receptor agonist or antagonist. In some embodiments, the ANS optimizing agent comprises benzodiazepine or a selective serotonin reuptake inhibitor (SSRI). In some embodiments, the ANS optimizing agent comprises one or more of lorazepam, clonazepam, pramipexole, ropinirole, rotigotine, apomorphine, trazodone, pregabalin, imipramine, clomipramine, amitriptyline, maprotiline, fluvoxamine, paroxetine, fluoxetine, milnacipran, chlorodiazepoxide, diazepam, estazolam, oxazepam, bromazepam, alprazolam, midazolam, clobazam, clotiazepam, quazepam, clorazepate, flurazepam, triazolam, temazepam, etizolam, trans-N-{4-[4-(2,3 -Dichlorophenyl)-1-piperazinyl]cyclohexyl}-3 -methoxybenzamide, (-)-7-{[2-(4-Phenylpiperazin-1-yl)ethyl]propylamino}-5,6,7,8-tetrahydronaphthalen-2-ol, 5-OH-DPAT, 7-OH-DPAT, 8-OH-PBZI (cis-8-Hydroxy-3-(n-propyl)-1,2,3a,4,5,9b-hexahydro-1H-benz[e]indole), Apomorphine, Bromocriptine, Captodiame, CJ-1639, Dopamine, ES609, FAUC 54, FAUC 73, PD-128,907, PF-219,06, PF-592,379, Piribedil, Pramipexole, Quinelorane, Quinpirole, Ropinirole, Rotigotine, Amisulpride, Cyproheptadine, PG 01037, Domperidone, FAUC 365, GR-103,691, GSK598809, Haloperidol, N-(4-(4-(2,3-Dichloro- or 2-methoxyphenyl)piperazin-1-yl)butyl)heterobiarylcarboxamides, Nafadotride, NGB-2904, PNU-99,194, Raclopride, S-14,297, S33084, SB-277011-A, SR 21502, Sulpiride, U99194, YQA14, Bradanicline, Encenicline, Tropisetron, Anabasine, Acetylcholine, Nicotine, Epiboxidine, Ivermectin, Galantamine, Anandamide, α-Bungarotoxin, α-Conotoxin,Bupropion, Dehydronorketamine Ethanol, Hydroxybupropion, Hydroxynorketamine, Ketamine, Kynurenic acid, Memantine, Lobeline, Methyllycaconitine, Norketamine, Quinolizidine and gaboxadol, isoguvacine, muscimol, progabide, piperidine-4-sulfonic acid Risperidone. In some embodiments, the ANS optimizing agent comprises one or more of lorazepam, clonazepam, pramipexole or trazodone.

In some embodiments, the treatment comprises hospitalization. In some embodiments, the treatment comprises treatment in an ICU and/or using ventilator support. In some embodiments, the treatment comprises administering to the subject a therapeutically effective amount of a therapeutic agent.

In some embodiments, the therapeutic agent comprises an anti-infection agent, an immunosuppressant and/or an immunomodulator. In some embodiments, the immunomodulator comprises one or more of azathioprine, cyclophosphamide, cyclosporine, hydroxychloroquine, leflunomide, methotrexate, mycophenolate, sulfasalazine, apremilast, tofacitinib, azathioprine, mercaptopurine, steroids, cortisone, cortisone acetate, dexamethasone, hydrocortisone, hydrocortisone acetate, methylprednisolone, prednisolone, prednisone, tixocortol pivalate, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, acleometasone dipropionate, betamethasone valerate, betamethasone dippropionate, prednicarbate, clobetasone-17-butyrate, clobetasol-17-propionate, fluocortilone caproate, fluocortolone pivalate, and fluprednidene acetate, hydrocortisone-17-butyrate, 17-aceponate, 17-buteprate, and prednicarbate. In some embodiments, the therapeutic agent comprises an anti-rheumatic drug. In some embodiments, the therapeutic agent comprises an IL-6 inhibitor. In some embodiments, the therapeutic agent comprises one or more of etanercept, infliximab, adalimumab, tocilizumab, rituximab, ofatumumab, belimumab, epratuzumab, abatacept, golimumab, certolizumab pegol, sifalimumab, anakinra, canakinumab, rilonacept, ruxolitinib, tofacitinib, oclacitinib, baricitinib, filgotinib, cucurbitacin gandotinib, lestaurtinib, momelotinib, pacritinib, pf-04965842, upadacitinib, peficitinib.

In some embodiments, the anti-infection agent comprises one or more of an anti-viral agent, antibiotic, anti-fungal agent, and an anti-parasitic agent. In some embodiments, the anti-viral agent comprises one or more of acyclovir, famcyclovir, sorivudine, trifluorothymidine, valacyclovir, dideoxyinosine, interferon alpha, lamivudine, rifampicin, baloxavir marboxil, famiciclovir, letermovir, Abacavir, Ziagen, Trizivir, Kivexa/Epzicom, Aciclovir, Acyclovir, Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Novir, Oseltamivir (Tamiflu), Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Sofosbuvir, Stavudine, Tea tree oil, Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir, and Zidovudine. In some embodiments, the antibiotic comprises one or more of penicillin, amoxicillin, ampicillin, hetacillin, cloxacillin, dicloxacillin, methicillin, nafcillin, oxacillin, axlocillin, carbenicillin, mezlocillin, piperacillin, ticarcillin, cefadroxil, cefazolin, cephalixin, cephalothin, cephapirin, cephradine, cefaclor, cefacmandole, cefmetazole, cefonicid, ceforanide, cefotetan, cefoxitin, cefprozil, cefuroxime, loracarbef, cefixime, cefoperazone, cefotaxime, cefpodoxime, ceftazidime, ceftiofur, ceftizoxime, ceftriaxone, moxalactam, aztreonam, imipenem, eropenem, ciprofloxacin, enrofloxacin, difloxacin, orbifloxacin, marbofloxacin, chloramphenicol, thiamphenicol, florfenicol, chlortetracycline, tetracycline, oxytetracycline, doxycycline, minocycline, erythromycin, tylosin, tlimicosin, clarithromycin, azithromycin, lincomycin, clindamycin, gentamicin, amikacin, kanamycin, apramycin, tobramycin, neomycin, dihydrostreptomycin, paromomycin, sulfadmethoxine, sfulfamethazine, sulfaquinoxaline, sulfamerazine, sulfathiazole, sulfasalazine, sulfadiazine, sulfabromomethazine, suflaethoxypyridazine, vancomycin, teicoplanin, ramoplanin, decaplanin, rifampin, nitrofuran, virginiamycin, polymyxins, and tobramycin. In some embodiments, the anti-fungal agent comprises one or more of itraconazole, ketoconazole, fluoconazole, voriconazole, griseofulvin and amphotericin B.

In some embodiments, the infectious disease comprises a cold, pneumonia, or influenza.

In some embodiments, the infectious disease comprises a viral infection, fungal infection, a bacterial infection, and/or a parasitic infection. In some embodiments, the infectious disease comprises an infection by one of more of a coronavirus, herpes simplex virus, papilloma virus, parainfluenza virus, influenza virus, hepatitis virus, Coxsackie Virus, herpes zoster virus, measles virus, mumps virus, rubella virus, rabies virus, hemorrhagic fever virus, human immunodeficiency virus (HIV), and H1N1 virus.

In some embodiments, the infectious disease comprises a coronavirus infection. In some embodiments, the coronavirus infection comprises an infection by a Middle East respiratory syndrome coronavirus (MERS-CoV), severe acute respiratory syndrome coronavirus (SARS-CoV), or SARS-CoV-2 (COVID-19).

In some embodiments, the infectious disease comprises an infection by one of more of Escherichia coli, Salmonella enterica, Shigella dysenteriae, Vibrio cholerae, Vibrio vulnificus, Vibrio parahaemolyticus, Virio vulnificus, Campylobacter jejuni, Klebsiella, Enterobacter, Serratia, Proteus, Providencia, and Morganella, Bacillus anthracis, Bacillus cereus, Clostridium tetani, Clostrium botulinum, Clostridium perfringens, Clostridium difficile, Mycobacterium tuberculosis, Legionella pneumophilla, Vibrio cholera, Staphylococcus aureus (such as Methicillin-resistant Staphylococcus aureus (MRSA)), Staphylococcus epidermidis, Staphylococcus saprophyticus, Streptococcus pyogenes, Streptococcus agalactiae, Enterococcus faecalis, Streptococcus bovis, Streptococcus pneumoniae, Streptococcus viridans, Pseudomonas aeruginosa, Corynebacterium diphtheriae, Listeria monocytogenes, Burcella, Francisella tularensis, Yersinia enterocolitica, Yersinia pseudotuberculosis, Yersinia pestis, Pasteurella multocida, Mycobacterium tuberculosis, Mycobacterium bovis, Mycobacterium avium, Mycobacterium leprae, Actinomyces israelii, Nocardia asteroides, Mycoplasma pneumoniae, Treponema pallidum, Borrelia brugdorferi, Leptospira interrogans, Chlamydia psittaci, Chlamydia trachomatis, Chlamydia pneumoniae, R. rickettsii, Coxiella burnetii, R. prowazekii, Gardnerella vaginalis, Lactobacillus, Peptococcus, Peptostreptococcus, Propionibacterium, Tropheryma, Burkholderia pseudomallei, and Burkholderia mallei.

In some embodiments, the infectious disease is associated with acute respiratory distress syndrome (ARDS) and/or a cytokine storm. In some embodiments, a targeted treatment for the infectious disease is not available. In some embodiments, a vaccine effective for the infectious disease is not available.

In some embodiments, the subject has acute respiratory distress syndrome (ARDS). In some embodiments, the subject has one or more comorbidities. In some embodiments, the one or more comorbidities comprise hypertension, diabetes and coronary heart disease. In some embodiments, the subject does not have a comorbidity.

### DETAILED DESCRIPTION

There are no targeted treatments yet for emerging infectious diseases, such as COVID-19. For example, 2-4% of COVID-19 infected patients can die from Acute Respiratory Distress Syndrome (ARDS) caused by an inflammatory cytokine storm. Currently, there are few insights as to why a specific patient develops cytokine storm, ARDS and death. Further, there are no effective options to address these severe symptoms of infectious diseases, such as COVID-19 infection, other than supportive care (e.g., O₂, ventilator support, etc.).

Provided herein are methods of treating a subject having an infectious disease, e.g., a viral disease. In general terms, the methods include co-administering to a subject having an infectious disease: an effective amount of an ANS optimizing therapy; and a treatment for the infection disease, wherein co-administering the ANS optimizing therapy improves a prognosis for treatment of the infectious disease. In some embodiments, the treatment is administered to the subject before, after, or concurrently to administering the ANS optimizing therapy. In some embodiments, the treatment is administered to the subject simultaneously with administering the ANS optimizing therapy. In some embodiments, the treatment and the ANS optimizing therapy are co-administered when one is administered within a time interval in which the other retains its physiological effect on the subject.

In some embodiments, co-administering the ANS optimizing therapy provides for an improved or favorable ANS profile in the subject (e.g., elevated parasympathetic activity levels and/or reduced sympathetic activity levels). Without being bound by theory, a favorable ANS profile (either as a categorical or continuous variable of HRV) is predictive of a less virulent infection and better outcome not requiring ANS optimization. In some embodiments, patients with a favorable ANS profile (high parasympathetic and low sympathetic activity) are treated with standard of infectious care. If upon screening (e.g., by measuring the heartbeat pattern and calculating the HRV), a patient having a favorable ANS profile can, in some embodiments, return home to convalesce, while another patient having the same level of symptomatology with poor ANS profile can be treated more aggressive, e.g., greater monitoring, quick hospitalization, faster advancement to more aggressive antibiotics (antivirals, etc.).

In some embodiments, the threshold values described herein are set by population percentages. For example, in some embodiments, a population of people is examined for the variable (e.g., HRV or other variable) and the resulting values determined across the population are divided into quartiles, with the top 25% percent being the most extreme level, the next 25% being moderate high, the next 25% being moderate low, and the final 25% being lowest for of the variable for the population. These values can then be used to establish cutoff values for particular actions for any single patient. In some embodiments, a depressed activity level is one that is lower than 50% of the population. In some embodiments, it is one that is in the lower 25% of the population or even lower 10% or even lower 5% of a population. In some embodiments, an elevated activity is one that is in the upper 50% of the population, e.g., upper 25%, upper 10%, or even upper 5% of a population. In some embodiments, the population is defined as a geographical population. In some embodiments, the population is one of at least 10 or more similarly defined subjects (e.g., same sex, age, and/or general fitness, etc.) In some embodiments, the population is at least 10, 20, 50, 100, 1000, or 10,000 or more subjects.

In some embodiments, those patients with a high likelihood of the disorder can be assigned such critical medical care equipment earlier, while those with a low likelihood need not receive such critical medical care. Such information can also be used to distribute medical equipment within the population or a hospital for example. Thus, in some embodiments, the prognosis of multiple patients can be used, in combination, to distribute device within a facility. This can also alter how much medical equipment may need to be purchased at any given time, by a care facility.

In some embodiments, the ANS optimizing therapy includes vagal nerve stimulation (VNS) and/or an ANS optimizing agent. VNS may be performed using any suitable option, e.g., to optimize the subject's ANS. In some embodiments, the VNS is delivered using an external or implantable device. In some embodiments, the VNS is delivered using a non-invasive technique. In some embodiments, an implantable VNS device delivers stimulation along any suitable position along the vagus nerve. Suitable VNS options include, without limitation, those provided by SetPoint Medical and Galvani Bioelectronics.

Without being bound by theory, it is thought the main ANS parasympathetic conduit, the vagus nerve, can impact immune function. Afferent vagal signals can alert the brain to inflammation, while efferent signals can reduce inflammation through activation of α-7 nicotinic acetylcholine receptors in the spleen, thereby reducing levels of proinflammatory cytokines, IL-1, IL-6 and TNF to reduce inflammation. Vagal nerve stimulation can reduce mouse inflammation to increased survival in a bacterial pathogen model (compared to controls). For example, transection of the vagus accelerated death in the same model. Thus, vagal nerve stimulation (VNS) can reduce inflammation in various conditions and diseases.

In some embodiments, the ANS optimizing therapy reduces sympathetic activity level of the subject. In some embodiments, the ANS optimizing therapy increases parasympathetic activity level of the subject. In some embodiments, the ANS optimizing therapy restores ANS balance in the subject.

Depending on the embodiment, a variety of ANS optimizing agents can be used in methods of the present disclosure. In some embodiments, the ANS optimizing agent includes, without limitation, one or more of an antidepressant, an anxiolytic, an anticonvulsant, a D₃ agonist or antagonist, a nicotinic acetylcholine receptor agonist or antagonist, an alpha-7 nicotinic receptor agonist or antagonist, a GABA_{A} receptor agonist or antagonist, an alpha-1 receptor agonist or antagonist, and an alpha-2 receptor agonist or antagonist. In some embodiments, the ANS optimizing agent includes, without limitation, one or more of benzodiazepine or a selective serotonin reuptake inhibitor (SSRI). In some embodiments, the ANS optimizing agent includes, without limitation, one or more of lorazepam, clonazepam, pramipexole, ropinirole, rotigotine, apomorphine, trazodone, pregabalin, imipramine, clomipramine, amitriptyline, maprotiline, fluvoxamine, paroxetine, fluoxetine, milnacipran, chlorodiazepoxide, diazepam, estazolam, oxazepam, bromazepam, alprazolam, midazolam, clobazam, clotiazepam, quazepam, clorazepate, flurazepam, triazolam, temazepam, etizolam, trans-N-{4-[4-(2,3 -Dichlorophenyl)-1-piperazinyl]cyclohexyl}-3 -methoxybenzamide, (-)-7-{[2-(4-Phenylpiperazin-1-yl)ethyl]propylamino}-5,6,7,8-tetrahydronaphthalen-2-ol, 5-OH-DPAT, 7-OH-DPAT, 8-OH-PBZI (cis-8-Hydroxy-3-(n-propyl)-1,2,3a,4,5,9b-hexahydro-1H-benz[e]indole), Apomorphine, Bromocriptine, Captodiame, CJ-1639, Dopamine, ES609, FAUC 54, FAUC 73, PD-128,907, PF-219,06, PF-592,379, Piribedil, Pramipexole, Quinelorane, Quinpirole, Ropinirole, Rotigotine, Amisulpride, Cyproheptadine, PG 01037, Domperidone, FAUC 365, GR-103,691, GSK598809, Haloperidol, N-(4-(4-(2,3-Dichloro- or 2-methoxyphenyl)piperazin-1-yl)butyl)heterobiarylcarboxamides, Nafadotride, NGB-2904, PNU-99,194, Raclopride, S-14,297, S33084, SB-277011-A, SR 21502, Sulpiride, U99194, YQA14, Bradanicline, Encenicline, Tropisetron, Anabasine, Acetylcholine, Nicotine, Epiboxidine, Ivermectin, Galantamine, Anandamide, a-Bungarotoxin, a-Conotoxin,Bupropion, Dehydronorketamine Ethanol, Hydroxybupropion, Hydroxynorketamine, Ketamine, Kynurenic acid, Memantine, Lobeline, Methyllycaconitine, Norketamine, Quinolizidine and gaboxadol, isoguvacine, muscimol, progabide, piperidine-4-sulfonic acid Risperidone. In some embodiments, the ANS optimizing agent includes, without limitation, one or more of lorazepam, clonazepam, pramipexole or trazodone.

Any suitable treatment for the infectious disease may be administered to the subject. In some embodiments, the treatment comprises hospitalization. In some embodiments, the subject is treated for the infectious disease in a medical facility, e.g., a hospital. In some embodiments, the treatment comprises treatment in an ICU and/or using ventilator support. In some embodiments, the subject is treated in an intensive care unit (ICU) and/or using ventilator support. In some embodiments, the subject is not treated in a medical facility, e.g., a hospital. In some embodiments, the subject is not treated in an intensive care unit (ICU) and/or using ventilator support.

In some embodiments, the treatment comprises administering to the subject a therapeutically effective amount of a therapeutic agent. Depending on the embodiment, a variety of therapeutic agents can be used in methods of the present disclosure. In some embodiments, the therapeutic agent includes, without limitation, one or more of an anti-infection agent, an immunosuppressant and/or an immunomodulatory. In some embodiments, the immunomodulator includes, without limitation, one or more of azathioprine, cyclophosphamide, cyclosporine, hydroxychloroquine, leflunomide, methotrexate, mycophenolate, sulfasalazine, apremilast, tofacitinib, azathioprine, mercaptopurine, steroids, cortisone, cortisone acetate, dexamethasone, hydrocortisone, hydrocortisone acetate, methylprednisolone, prednisolone, prednisone, tixocortol pivalate, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, acleometasone dipropionate, betamethasone valerate, betamethasone dippropionate, prednicarbate, clobetasone-17-butyrate, clobetasol-17-propionate, fluocortilone caproate, fluocortolone pivalate, and fluprednidene acetate, hydrocortisone-17-butyrate, 17-aceponate, 17-buteprate, and prednicarbate.

In some embodiments, the therapeutic agent includes an anti-rheumatic drug. In some embodiments, the therapeutic agent includes an IL-6 inhibitor. In some embodiments, the therapeutic agent includes, without limitation, one or more of etanercept, infliximab, adalimumab, tocilizumab, rituximab, ofatumumab, belimumab, epratuzumab, abatacept, golimumab, certolizumab pegol, sifalimumab, anakinra, canakinumab, rilonacept, ruxolitinib, tofacitinib, oclacitinib, baricitinib, filgotinib, cucurbitacin gandotinib, lestaurtinib, momelotinib, pacritinib, pf-04965842, upadacitinib, peficitinib.

In some embodiments, the anti-infection agent includes, without limitation, one or more of an anti-viral agent, antibiotic, anti-fungal agent, and an anti-parasitic agent. In some embodiments, the anti-viral agent includes, without limitation, one or more of acyclovir, famcyclovir, sorivudine, trifluorothymidine, valacyclovir, dideoxyinosine, interferon alpha, lamivudine, rifampicin, baloxavir marboxil, famiciclovir, letermovir, Abacavir, Ziagen, Trizivir, Kivexa/Epzicom, Aciclovir, Acyclovir, Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Novir, Oseltamivir (Tamiflu), Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Sofosbuvir, Stavudine, Tea tree oil, Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir, and Zidovudine. In some embodiments, the antibiotic includes, without limitation, one or more of penicillin, amoxicillin, ampicillin, hetacillin, cloxacillin, dicloxacillin, methicillin, nafcillin, oxacillin, axlocillin, carbenicillin, mezlocillin, piperacillin, ticarcillin, cefadroxil, cefazolin, cephalixin, cephalothin, cephapirin, cephradine, cefaclor, cefacmandole, cefmetazole, cefonicid, ceforanide, cefotetan, cefoxitin, cefprozil, cefuroxime, loracarbef, cefixime, cefoperazone, cefotaxime, cefpodoxime, ceftazidime, ceftiofur, ceftizoxime, ceftriaxone, moxalactam, aztreonam, imipenem, eropenem, ciprofloxacin, enrofloxacin, difloxacin, orbifloxacin, marbofloxacin, chloramphenicol, thiamphenicol, florfenicol, chlortetracycline, tetracycline, oxytetracycline, doxycycline, minocycline, erythromycin, tylosin, tlimicosin, clarithromycin, azithromycin, lincomycin, clindamycin, gentamicin, amikacin, kanamycin, apramycin, tobramycin, neomycin, dihydrostreptomycin, paromomycin, sulfadmethoxine, sfulfamethazine, sulfaquinoxaline, sulfamerazine, sulfathiazole, sulfasalazine, sulfadiazine, sulfabromomethazine, suflaethoxypyridazine, vancomycin, teicoplanin, ramoplanin, decaplanin, rifampin, nitrofuran, virginiamycin, polymyxins, and tobramycin. In some embodiments, the anti-fungal agent include, without limitation, one or more of itraconazole, ketoconazole, fluoconazole, voriconazole, griseofulvin and amphotericin B.

An “effective amount” or “therapeutically effective amount” can be a quantity of a therapeutic agent or ANS optimizing agent sufficient to achieve a desired effect in a subject being treated. For instance, this can be the amount necessary to fully ameliorate, prevent, reduce or inhibit one or more signs or symptoms associated with an infectious disease, or delays the onset of one or more of the symptoms of a particular disease or condition. In some embodiments, the effective amount is sufficient to alter one or more of the other variables being monitored for the prognosis, such as HRV or ANS. Such amount can be administered as a single dosage or can be administered according to a regimen, whereby it is effective. Repeated administration may be needed to achieve a desired result (e.g., treatment of the disease and/or condition).

The disclosed agents and compounds may be used alone or in combination with other treatments. These agents or compounds, when used in combination with other agents, may be administered as a daily dose or an appropriate fraction of the daily dose (e.g., bid). The compounds may be administered after a course of treatment by another agent, during a course of therapy with another agent, administered as part of a therapeutic regimen, or may be administered prior to therapy by another agent in a treatment program.

Examples of pharmaceutically acceptable salts include acetate, adipate, besylate, bromide, camsylate, chloride, citrate, edisylate, estolate, fumarate, gluceptate, gluconate, glucuronate, hippurate, hyclate, hydrobromide, hydrochloride, iodide, isethionate, lactate, lactobionate, maleate, mesylate, methylbromide, methylsulfate, napsylate, nitrate, oleate, palmoate, phosphate, polygalacturonate, stearate, succinate, sulfate, sulfosalicylate, tannate, tartrate, terphthalate, tosylate, and triethiodide.

Compositions containing the active ingredient may be in any form suitable for the intended method of administration. Suitable routes of administration include, for example, oral, rectal, topical, nasal, pulmonary, ocular, intestinal, and parenteral administration. Suitable routes of parenteral administration include, without limitation, intravenous, intramuscular, and subcutaneous administration. Other suitable routes of administration include, without limitation, intraperitoneal, intra-arterial, intra-articular, intracardiac, intracisternal, intradermal, intralesional, intraocular, intrapleural, intrathecal, intrauterine, and intraventricular administration. The infectious disease to be treated, along with the physical, chemical, and biological properties of the agent being administered, can dictate the type of formulation and the route of administration to be used, as well as whether local or systemic delivery would be preferred.

When the compounds are administered via oral administration, for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient can be mixed with an inert solid diluent, for example calcium phosphate or kaolin, or as soft gelatin capsules wherein the active ingredient can be mixed with water or an oil medium, such as peanut oil, liquid paraffin or olive oil.

Formulations suitable for parenteral administration include aqueous and non-aqueous isotonic sterile injection solutions which may contain, for example, antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Injection solutions and suspensions may be prepared from sterile powders, granules and tablets of the kind previously described.

In some embodiments unit dosage formulations contain a daily dose or unit, daily sub-dose, or an appropriate fraction thereof, of a drug. It will be understood, however, that the specific dose level for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those skilled in the art.

The present embodiments include pharmaceutical formulations comprising one or more compounds described throughout in association with a pharmaceutically acceptable carrier. Preferably these formulations are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual, buccal, topical or rectal administration, or for administration by inhalation or insufflation. Also, the instant compounds can be administered to the body through Xenoport technology. XenoPort identifies and characterizes transporters throughout the body that are useful to drug delivery, then uses selected transporter proteins as “targets” and employs medicinal chemistry techniques to modify drugs into substrates for these transporters.

Alternatively, the formulations may be presented in a form suitable for once-daily, once-weekly or once-monthly administration; for example, an insoluble salt of the active compound may be adapted to provide a preparation for intramuscular injection. The pharmaceutical formulations described herein can be administered to a patient per se, or in pharmaceutical formulations where they are mixed with other active ingredients, as in combination therapy, or suitable pharmaceutically acceptable carriers or excipient(s). Techniques for formulation and administration of the compounds of the instant application may be found in “Remington's Pharmaceutical Sciences,” Mack Publishing Co., Easton, Pa., 18th edition, 1990.

For preparing solid pharmaceutical formulations such as tablets, the principal active ingredient is mixed with a pharmaceutically acceptable carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation formulation containing a homogeneous mixture of a compound of the present embodiments, or a pharmaceutically acceptable salt thereof. When referring to these preformulation formulations as homogeneous, it is meant that the active ingredient is dispersed evenly throughout the formulation so that the formulation may be readily subdivided into equally effective unit dosage forms such as tablets, pills and capsules. This solid preformulation formulation is then subdivided into unit dosage forms of the type described above containing from about 0.01 to about 10,000 mg of the compounds of the present embodiments. Preferably the dosage is from about 50 to about 5000 mg; more preferably, the dosage is from about 450 to about 1800 mg; even more preferably, the dosage is from about 600 to about 1000 mg. The tablets or pills of the novel formulation can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Furthermore, compounds for the present embodiments can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

Pharmaceutical formulations for parenteral administration, e.g., by bolus injection or continuous infusion, include aqueous solutions of the active compounds in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The formulations may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For oral administration, the instant compounds can be formulated readily by combining the active compounds with pharmaceutically acceptable carriers well known in the art. Such pharmaceutically acceptable carriers enable the compounds of the present embodiments to be formulated as tablets, pills, dragees, capsules, liquids, gels, syrups, slurries, suspensions and the like, for oral ingestion by a patient to be treated. Pharmaceutical formulations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

For buccal administration, the pharmaceutical formulations may take the form of tablets, lozenges, wafers and rapid-dissolve preparations formulated in conventional manner.

The compounds of the present embodiments can also be administered in the form of liposome pharmaceutical formulations, such as small unilamellar vesicles, large unilamellar vesicles and multilamellar vesicles. Liposomes can be formed from a variety of phospholipids, such as cholesterol, stearylamine or phosphatidylcholines.

Further disclosed herein are various pharmaceutical formulations well known in the pharmaceutical art for uses that include intraocular, intranasal, and intraauricular delivery. Suitable penetrants for these uses are generally known in the art. Pharmaceutical formulations for intraocular delivery include aqueous ophthalmic solutions of the active compounds in water-soluble form, such as eyedrops, or in gellan gum (Shedden et al., Clin. Ther., 23(3):440-50 (2001)) or hydrogels (Mayer et al., Ophthalmologica, 210(2):101-3 (1996)); ophthalmic ointments; ophthalmic suspensions, such as microparticulates, drug-containing small polymeric particles that are suspended in a liquid carrier medium (Joshi, A., J. Ocul. Pharmacol., 10(1):29-45 (1994)), lipid-soluble formulations (Alm et al., Prog. Clin. Biol. Res., 312:447-58 (1989)), and microspheres (Mordenti, Toxicol. Sci., 52(1):101-6 (1999)); and ocular inserts.

Alternatively, other delivery systems for hydrophobic pharmaceutical compounds may be employed. Liposomes and emulsions are well known examples of delivery vehicles or pharmaceutical acceptable carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The dosage regimen utilizing the compounds of the present embodiments is selected in accordance with a variety of factors including type, species, age, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the pharmaceutical formulation's availability to target sites. This involves a consideration of the distribution, equilibrium, and elimination of the compounds. Advantageously, compounds of the present embodiments may be administered, for example, in a single daily dose, or the total daily dosage may be administered in divided doses of two, three or four times daily.

In the methods of the present embodiments, the pharmaceutical formulations herein described in detail are typically administered in accordance with conventional pharmaceutical practices.

For instance, for oral administration in the form of a tablet or capsule, the compounds of the present embodiments can be combined with an oral, non-toxic pharmaceutically acceptable inert carrier such as ethanol, glycerol, water and the like. Moreover, when desired or necessary, suitable pharmaceutically acceptable carriers, such as, binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Some examples of pharmaceutically acceptable carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety.

The oral liquid formulations in which the present embodiments may be incorporated for administration orally include using pharmaceutically acceptable carriers, aqueous solutions, suitably flavoured syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous oral suspensions include synthetic and natural gums such as tragacanth, acacia, alginate, dextran, sodium carboxymethylcellulose, methylcellulose, polyvinyl-pyrrolidone or gelatin. Other dispersing agents which may be employed include glycerin and the like.

The daily dosage of the products may be varied over a wide range; e.g., from about 10 to about 10,000 mg per adult human per day. For oral administration, the formulations are preferably provided in the form of tablets containing about 0.001, 0.01, 0.1, 1, 10.0, 15.0, 25.0, 50.0, 100, 200, 300, 400, 500, 600, 700, 800, 900 1000, 2000, 3000, 4000, 5000, 6000, 7000, 8000, 9000 or 10,000 milligrams of the active ingredient for the symptomatic adjustment of the dosage to the patient to be treated. The instant pharmaceutical formulations typically contain from 10 mg to about 2000 mg of the instant compounds, preferably, from about 50 mg to about 1000 mg of active ingredient. An effective amount of the instant compounds is ordinarily supplied at a dosage level of from about 0.002 mg/kg to about 150 mg/kg of body weight per day. Preferably, the range is from about 0.02 to about 80 mg/kg of body weight per day, and especially from about 0.2 mg/kg to about 40 mg/kg of body weight per day. The compounds may be administered on a regimen of about 1 to about 10 times per day.

The subject in the various methods of the present disclosure can be any suitable subject having an infectious disease. In some embodiments, the subject is a mammal (e.g., mouse, rat, dog, cat, horse, pig, cow, camel, rabbit, sheep, primate, including non-human primate, or human). In some embodiments, the subject has acute respiratory distress syndrome (ARDS). In some embodiments, the subject has developed ARDS as a consequence of having the infectious disease. In some embodiments, the subject has one or more comorbidities. The subject may have a variety of comorbidities, depending on the embodiment. In some embodiments, the subject has one or more of hypertension, diabetes and coronary heart disease. In some embodiments, the subject does not have a comorbidity.

According to some embodiments, the subject has one or more of a variety of infectious diseases. In some embodiments, the infectious disease includes, without limitation, a cold, pneumonia, or influenza. In some embodiments, the infectious disease is associated with acute respiratory distress syndrome (ARDS) and/or a cytokine storm. In some embodiments, a subject having the infectious disease has a chance of developing ARDS and/or a cytokine storm of about 1% or more, 2% or more, 3% or more, 4% or more, 5% or more, 10% or more, 15% or more, 20% or more, including 25% or more, or a percentage within a range defined by any two of the preceding values. In some embodiments, a subject having the infectious disease and one or more comorbidities (e.g., high blood pressure, diabetes, coronary heart disease, etc.) has a chance of developing ARDS and/or a cytokine storm of about 1% or more, 2% or more, 3% or more, 4% or more, 5% or more, 10% or more, 15% or more, 20% or more, including 25% or more, or a percentage within a range defined by any two of the preceding values. In some embodiments, the infectious disease increases the chances of the subject having the disease developing acute respiratory distress syndrome (ARDS) and/or a cytokine storm.

In some embodiments, a targeted treatment for the infectious disease is not available. In some embodiments, a targeted treatment for the infectious disease is not available when there is no therapeutic agent known or available to specifically target the infectious agent (e.g., virus, bacteria, fungus, parasite, etc.) to treat the infectious disease. In some embodiments, a targeted treatment for the infectious disease is not available when there is no vaccine effective against the infectious agent causing the infectious disease is available. In some embodiments, a targeted treatment for the infectious disease is not available when there is no antibiotic, anti-viral, anti-fungal, or anti-parasitic agent effective against the infectious agent causing the infectious disease is available.

In some embodiments, the infectious disease includes, without limitation, a viral infection, fungal infection, a bacterial infection, and/or a parasitic infection. In some embodiments, the infectious disease includes, without limitation, an infection by one of more of a coronavirus, herpes simplex virus, papilloma virus, parainfluenza virus, influenza virus, hepatitis virus, Coxsackie Virus, herpes zoster virus, measles virus, mumps virus, rubella virus, rabies virus, hemorrhagic fever virus, human immunodeficiency virus (HIV) and H1N1 virus.

In some embodiments, the infectious disease the infectious disease is a coronavirus infection. In some embodiments, the coronavirus infection includes an infection by, without limitation, a Middle East respiratory syndrome coronavirus (MERS-CoV), severe acute respiratory syndrome coronavirus (SARS-CoV), or SARS-CoV-2 (COVID-19).

In some embodiments, the infectious disease includes, without limitation, an infection by one of more of Escherichia coli, Salmonella enterica, Shigella dysenteriae, Vibrio cholerae, Vibrio vulnificus, Vibrio parahaemolyticus, Virio vulnificus, Campylobacter jejuni, Klebsiella, Enterobacter, Serratia, Proteus, Providencia, and Morganella, Bacillus anthracis, Bacillus cereus, Clostridium tetani, Clostrium botulinum, Clostridium perfringens, Clostridium difficile, Mycobacterium tuberculosis, Legionella pneumophilla, Vibrio cholera, Staphylococcus aureus (such as Methicillin-resistant Staphylococcus aureus (MRSA)), Staphylococcus epidermidis, Staphylococcus saprophyticus, Streptococcus pyogenes, Streptococcus agalactiae, Enterococcus faecalis, Streptococcus bovis, Streptococcus pneumoniae, Streptococcus viridans, Pseudomonas aeruginosa, Corynebacterium diphtheriae, Listeria monocytogenes, Burcella, Francisella tularensis, Yersinia enterocolitica, Yersinia pseudotuberculosis, Yersinia pestis, Pasteurella multocida, Mycobacterium tuberculosis, Mycobacterium bovis, Mycobacterium avium, Mycobacterium leprae, Actinomyces israelii, Nocardia asteroides, Mycoplasma pneumoniae, Treponema pallidum, Borrelia brugdorferi, Leptospira interrogans, Chlamydia psittaci, Chlamydia trachomatis, Chlamydia pneumoniae, R. rickettsii, Coxiella burnetii, R. prowazekii, Gardnerella vaginalis, Lactobacillus, Peptococcus, Peptostreptococcus, Propionibacterium, Tropheryma, Burkholderia pseudomallei, and Burkholderia mallei.

In some embodiments, the method includes testing the subject for the infectious disease. The subject may be tested for the infectious disease at any suitable time. In some embodiments, testing for the infectious disease is performed before, after, or concurrently to measuring the HRV. Testing for the infectious disease can be performed using any suitable option. In some embodiments, testing includes a clinical observation and/or diagnostic testing.

As used herein, “prognosis” has its ordinary and customary meaning, in view of the present disclosure. “Prognosis” can refer to one or more potential outcomes for a condition or disease of a subject (e.g., a patient) at one or more points or periods of time in the future. The likelihood of the outcome in a prognosis may vary depending on the embodiment. In some embodiments, the prognosis is a likely outcome. In some embodiments, the prognosis is a reasonably certain outcome. In some embodiments, the likelihood of the outcome is 10% or more, e.g., 20% or more, 30% or more, 40% or more, 50% or more, 60% or more, 70% or more, 80% or more, 90% or more, 95% or more, including about 100%, or any percentage within a range defined by any two of the preceding values. In some embodiments, the likelihood of an outcome is expressed as an odds ratio. In some embodiments, methods of the present disclosure improves the odds ratio of a desirable outcome for the treatment of the infectious disease.

In some embodiments, the prognosis includes one or more of chance of survival, ventilator time, intensive care unit (ICU) time, hospitalization time, time to recovery, rehabilitation time, back-to-employment time, permanent disability, near-term and/or extended-term mortality rate. In some embodiments, the treatment for the infectious disease comprises treatment of the subject in an intensive care unit (ICU) and/or using ventilator support. The prognosis can include an outcome for one or more of a variety of treatments of the infectious disease, as described herein.

In general, the prognosis is improved by the present methods when compared to a treatment of the infectious disease without co-administering the ANS optimizing therapy. In some embodiments, the prognosis is improved by the present methods when compared to the prognosis of an average patient with the infectious disease, e.g., an average patient to whom the ANS optimizing therapy is not co-administered. In some embodiments, the prognosis is improved when compared to an average patient with the infectious disease, e.g., an average patient to whom the ANS optimizing therapy is not co-administered, and having the same clinical profile as the subject. In some embodiments, the prognosis is improved when compared to an average patient with the infectious disease, e.g., an average patient to whom the ANS optimizing therapy is not co-administered, and having the same comorbidities as the subject. In some embodiments, the prognosis is improved when compared to the prognosis before co-administering the ANS optimizing therapy to the subject. In some embodiments, an improved prognosis includes complete or partial recovery from the infectious disease without the need for further medical intervention, e.g., without the need for further hospitalization. In some embodiments, an improved prognosis includes complete or partial recovery from the infectious disease without the need for further resource-intensive treatment for the infectious disease, e.g., without the need for further treatment in an intensive care unit (ICU) and/or continued use of ventilator support.

In some embodiments, an improved prognosis includes a reduced risk of suffering a cytokine storm due to the infectious disease. In some embodiments, the risk of suffering a cytokine storm due to the infectious disease is reduced by at least about 5%, at least about 10%, at least about 20%, at least about 30%, at least about 40%, at least about 50%, at least about 60%, at least about 70%, at least about 80%, at least about 90%, at least about 95%, or about 100%, or by any percentage within a range defined by any two of the preceding values. In some embodiments, an improved prognosis includes a reduced risk of suffering from ARDS due to the infectious disease. In some embodiments, the risk of suffering from ARDS due to the infectious disease is reduced by at least about 5%, at least about 10%, at least about 20%, at least about 30%, at least about 40%, at least about 50%, at least about 60%, at least about 70%, at least about 80%, at least about 90%, at least about 95%, or about 100%, or by any percentage within a range defined by any two of the preceding values.

In some embodiments, an improved prognosis includes an increased chance of survival compared to a reference percentage (e.g., an average chance of survival for a patient with the infectious disease, an average chance of survival for a patient with the infectious disease and having the same clinical profile as the subject, an average chance of survival for a patient with the infectious disease and having the same comorbidities as the subject, etc.). In some embodiments, an improved prognosis includes a chance of survival that is increased compared to the chance of survival before co-administering the ANS optimizing therapy, or to the chance of survival with treatment for the infectious disease but without co-administering the ANS optimizing therapy.

In some embodiments, an improved prognosis includes a reduced near-term mortality compared to a reference near-term mortality (e.g., an average near-term mortality for a patient with the infectious disease, an average near-term mortality for a patient with the infectious disease and having the same clinical profile as the subject, an average near-term mortality for a patient with the infectious disease and having the same comorbidities as the subject, etc.). In some embodiments, an improved prognosis includes a generally desirably low near-term mortality. In some embodiments, an improved prognosis includes a near-term mortality that is reduced compared to the near-term mortality before co-administering the ANS optimizing therapy, or to the near-term mortality with treatment for the infectious disease but without co-administering the ANS optimizing therapy.

In some embodiments, an improved prognosis includes a reduced extended-term mortality compared to a reference extended-term mortality (e.g., an average extended-term mortality for a patient with the infectious disease, an average extended-term mortality for a patient with the infectious disease and having the same clinical profile as the subject, an average extended-term mortality for a patient with the infectious disease and having the same comorbidities as the subject, etc.). In some embodiments, an improved prognosis includes a generally desirably low extended-term mortality. In some embodiments, an improved prognosis includes an extended-term mortality that is reduced compared to the extended-term mortality before co-administering the ANS optimizing therapy, or to the extended-term mortality with treatment for the infectious disease but without co-administering the ANS optimizing therapy.

In some embodiments, an improved prognosis includes a reduced risk of permanent disability compared to a reference risk of permanent disability (e.g., an average risk of permanent disability for a patient with the infectious disease, an average risk of permanent disability for a patient with the infectious disease and having the same clinical profile as the subject, an average risk of permanent disability for a patient with the infectious disease and having the same comorbidities as the subject, etc.). In some embodiments, an improved prognosis includes a generally desirably low risk of permanent disability. In some embodiments, an improved prognosis includes a risk of permanent disability that is reduced compared to the risk of permanent disability before co-administering the ANS optimizing therapy, or to the risk of permanent disability with treatment for the infectious disease but without co-administering the ANS optimizing therapy.

In some embodiments, an improved prognosis includes reduced ventilator time (e.g., time during which ventilator support is required) compared to a reference ventilator time (e.g., an average ventilator time for a patient with the infectious disease, an average ventilator time for a patient with the infectious disease and having the same clinical profile as the subject, an average ventilator time for a patient with the infectious disease and having the same comorbidities as the subject, etc.). In some embodiments, an improved prognosis includes a generally desirably short ventilator time. In some embodiments, an improved prognosis includes a ventilator time that is reduced compared to the ventilator time before co-administering the ANS optimizing therapy, or to the ventilator time with treatment for the infectious disease but without co-administering the ANS optimizing therapy.

In some embodiments, an improved prognosis includes reduced ICU time (e.g., time spent in the ICU) compared to a reference ICU time (e.g., an average ICU time for a patient with the infectious disease, an average ICU time for a patient with the infectious disease and having the same clinical profile as the subject, an average ICU time for a patient with the infectious disease and having the same comorbidities as the subject, etc.). In some embodiments, an improved prognosis includes a generally desirably short ICU time. In some embodiments, an improved prognosis includes an ICU time that is reduced compared to the ICU time before co-administering the ANS optimizing therapy, or to the ICU time with treatment for the infectious disease but without co-administering the ANS optimizing therapy.

In some embodiments, an improved prognosis includes reduced hospitalization time (e.g., time during which hospital care is required) compared to a reference hospitalization time (e.g., an average hospitalization time for a patient with the infectious disease, an average hospitalization time for a patient with the infectious disease and having the same clinical profile as the subject, an average hospitalization time for a patient with the infectious disease and having the same comorbidities as the subject, etc.). In some embodiments, an improved prognosis includes a generally desirably short hospitalization time. In some embodiments, an improved prognosis includes a hospitalization time that is reduced compared to the hospitalization time before co-administering the ANS optimizing therapy, or to the hospitalization time with treatment for the infectious disease but without co-administering the ANS optimizing therapy.

In some embodiments, an improved prognosis includes reduced time to recovery compared to a reference time to recovery (e.g., an average time to recovery for a patient with the infectious disease, an average time to recovery for a patient with the infectious disease and having the same clinical profile as the subject, an average time to recovery for a patient with the infectious disease and having the same comorbidities as the subject, etc.). In some embodiments, an improved prognosis includes a generally desirably short time to recovery for a patient having the infectious disease. In some embodiments, an improved prognosis includes a time to recovery that is reduced compared to the time to recovery before co-administering the ANS optimizing therapy, or to the time to recovery with treatment for the infectious disease but without co-administering the ANS optimizing therapy.

In some embodiments, an improved prognosis includes reduced rehabilitation time compared to a reference rehabilitation time (e.g., an average rehabilitation time for a patient treated for the infectious disease, an average rehabilitation time for a patient treated for the infectious disease and having the same clinical profile as the subject, an average rehabilitation time for a patient treated for the infectious disease and having the same comorbidities as the subject, etc.). In some embodiments, an improved prognosis includes a generally desirably short rehabilitation time for a patient with the infectious disease. In some embodiments, an improved prognosis includes a rehabilitation time that is reduced compared to the rehabilitation time before co-administering the ANS optimizing therapy, or to the rehabilitation time with treatment for the infectious disease but without co-administering the ANS optimizing therapy.

In some embodiments, an improved prognosis includes reduced back-to-employment time (e.g., time from when the subject becomes unable to continue in his/her regular course of employment because the infectious disease until the subject regains his/her employment status) compared to a reference back-to-employment time (e.g., an average back-to-employment time for a patient with the infectious disease, an average back-to-employment time for a patient with the infectious disease and having the same clinical profile as the subject, an average back-to-employment time for a patient with the infectious disease and having the same comorbidities as the subject, etc.). In some embodiments, an improved prognosis includes a generally desirably short back-to-employment time for a patient with the infectious disease. In some embodiments, an improved prognosis includes a back-to-employment that is reduced compared to the back-to-employment before co-administering the ANS optimizing therapy, or to the back-to-employment with treatment for the infectious disease but without co-administering the ANS optimizing therapy.

In some embodiments, an improved prognosis indicates an outcome improved by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, or by a percentage within a range defined by any two of the preceding values, compared to a reference outcome. For example, in some embodiments, an improved prognosis includes a chance of survival that is increased by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, or by a percentage within a range defined by any two of the preceding values, compared to a reference percentage. For example, in some embodiments, an improved prognosis includes a ventilator time reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, about 100%, or by a percentage within a range defined by any two of the preceding values, compared to a reference ventilator time. In some embodiments, an improved prognosis indicates an outcome improved by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, or by a percentage within a range defined by any two of the preceding values, compared to the outcome for the subject before receiving treatment for the infectious disease.

Additional embodiments of the present disclosure are described. In some embodiments, any option for ANS optimization (such as, but not limited to, drugs for treating restless leg syndrome (RLS), VNS, and other therapeutic agents having favorable ANS impact) can be used to improve outcomes for pandemic infectious disease, such as, but not limited to, COVID-19. In some embodiments, the improved outcomes include, without limitation, improved survival rate, hospitalization time, ICU time, cost of care, etc.

Some embodiments of the present disclosure are further provided in the following numbered arrangements.

1. A method of treating a subject having an infectious disease, comprising co-administering to a subject having an infectious disease:
a therapeutically effective amount of an ANS optimizing therapy; and
a treatment for the infectious disease,
wherein co-administering the ANS optimizing therapy improves the prognosis for treatment of the infectious disease.

2. The method of arrangement 1, wherein the ANS optimizing therapy and the treatment are administered concurrently.

3. The method of arrangement 1, wherein the ANS optimizing therapy and the treatment are administered simultaneously.

4. The method of arrangement 1, wherein the ANS optimizing therapy is administered before administering the treatment.

5. The method of arrangement 1, wherein the ANS optimizing therapy is administered after administering the treatment.

6. The method of any one of the preceding arrangements, comprising testing the subject for the infectious disease before co-administering.

7. The method of arrangement 6, wherein testing is performed before co-administering.

8. The method of any one of the preceding arrangements, wherein the ANS optimizing therapy comprises one or more of VNS, and/or an ANS optimizing agent.

9. The method of arrangement 8, wherein the ANS optimizing agent comprises an antidepressant, an anxiolytic, an anticonvulsant, a D₃ agonist or antagonist, a nicotinic acetylcholine receptor agonist or antagonist, an alpha-7 nicotinic receptor agonist or antagonist, a GABA_{A} receptor agonist or antagonist, an alpha-1 receptor agonist or antagonist, and an alpha-2 receptor agonist or antagonist.

10. The method of arrangement 8, wherein the ANS optimizing agent comprises benzodiazepine or a selective serotonin reuptake inhibitor (SSRI).

11. The method of arrangement 8, wherein the ANS optimizing agent comprises one or more of lorazepam, clonazepam, pramipexole, ropinirole, rotigotine, apomorphine, trazodone, pregabalin, imipramine, clomipramine, amitriptyline, maprotiline, fluvoxamine, paroxetine, fluoxetine, milnacipran, chlorodiazepoxide, diazepam, estazolam, oxazepam, bromazepam, alprazolam, midazolam, clobazam, clotiazepam, quazepam, clorazepate, flurazepam, triazolam, temazepam, etizolam, trans-N-{4-[4-(2,3 -Dichlorophenyl)-1-piperazinyl]cyclohexyl}-3 -methoxybenzamide, (-)-7-{[2-(4-Phenylpiperazin-1-yl)ethyl]propylamino}-5,6,7,8-tetrahydronaphthalen-2-ol, 5-OH-DPAT, 7-OH-DPAT, 8-OH-PBZI (cis-8-Hydroxy-3-(n-propyl)-1,2,3a,4,5,9b-hexahydro-1H-benz[e]indole), Apomorphine, Bromocriptine, Captodiame, CJ-1639, Dopamine, ES609, FAUC 54, FAUC 73, PD-128,907, PF-219,06, PF-592,379, Piribedil, Pramipexole, Quinelorane, Quinpirole, Ropinirole, Rotigotine, Amisulpride, Cyproheptadine, PG 01037, Domperidone, FAUC 365, GR-103,691, GSK598809, Haloperidol, N-(4-(4-(2,3-Dichloro- or 2-methoxyphenyl)piperazin-1-yl)butyl)heterobiarylcarboxamides, Nafadotride, NGB -2904, PNU-99,194, Raclopride, S-14,297, S33084, SB-277011-A, SR 21502, Sulpiride, U99194, YQA14, Bradanicline, Encenicline, Tropisetron, Anabasine, Acetylcholine, Nicotine, Epiboxidine, Ivermectin, Galantamine, Anandamide, α-Bungarotoxin, α-Conotoxin,Bupropion, Dehydronorketamine Ethanol, Hydroxybupropion, Hydroxynorketamine, Ketamine, Kynurenic acid, Memantine, Lobeline, Methyllycaconitine, Norketamine, Quinolizidine and gaboxadol, isoguvacine, muscimol, progabide, piperidine-4-sulfonic acid Risperidone.

12. The method of arrangement 8, wherein the ANS optimizing agent comprises one or more of lorazepam, clonazepam, pramipexole or trazodone.

13. The method of any one of the preceding arrangements, wherein the treatment comprises hospitalization.

14. The method of any one of the preceding arrangements, wherein the treatment comprises treatment in an ICU and/or using ventilator support.

15. The method of any one of the preceding arrangements, wherein the treatment comprises administering to the subject a therapeutically effective amount of a therapeutic agent.

16. The method of arrangement 15, wherein the therapeutic agent comprises an anti-infection agent, an immunosuppres sant and/or an immunomodulator.

17. The method of arrangement 16, wherein the immunomodulator comprises one or more of azathioprine, cyclophosphamide, cyclosporine, hydroxychloroquine, leflunomide, methotrexate, mycophenolate, sulfasalazine, apremilast, tofacitinib, azathioprine, mercaptopurine, steroids, cortisone, cortisone acetate, dexamethasone, hydrocortisone, hydrocortisone acetate, methylprednisolone, prednisolone, prednisone, tixocortol pivalate, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, acleometasone dipropionate, betamethasone valerate, betamethasone dippropionate, prednicarbate, clobetasone-17-butyrate, clobetasol-17-propionate, fluocortilone caproate, fluocortolone pivalate, and fluprednidene acetate, hydrocortisone-17-butyrate, 17-aceponate, 17-buteprate, and prednicarbate.

18. The method of arrangement 15, wherein the therapeutic agent comprises an anti-rheumatic drug.

19. The method of arrangement 15, wherein the therapeutic agent comprises an IL-6 inhibitor.

20. The method of arrangement 15, wherein the therapeutic agent comprises one or more of etanercept, infliximab, adalimumab, tocilizumab, rituximab, ofatumumab, belimumab, epratuzumab, abatacept, golimumab, certolizumab pegol, sifalimumab, anakinra, canakinumab, rilonacept, ruxolitinib, tofacitinib, oclacitinib, baricitinib, filgotinib, cucurbitacin gandotinib, lestaurtinib, momelotinib, pacritinib, pf-04965842, upadacitinib, peficitinib.

21. The method of arrangement 15, wherein the anti-infection agent comprises one or more of an anti-viral agent, antibiotic, anti-fungal agent, and an anti-parasitic agent.

22. The method of arrangement 21, wherein the anti-viral agent comprises one or more of acyclovir, famcyclovir, sorivudine, trifluorothymidine, valacyclovir, dideoxyinosine, interferon alpha, lamivudine, rifampicin, baloxavir marboxil, famiciclovir, letermovir, Abacavir, Ziagen, Trizivir, Kivexa/Epzicom, Aciclovir, Acyclovir, Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Novir, Oseltamivir (Tamiflu), Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Sofosbuvir, Stavudine, Tea tree oil, Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir, and Zidovudine.

23. The method of arrangement 21, wherein the antibiotic comprises one or more of penicillin, amoxicillin, ampicillin, hetacillin, cloxacillin, dicloxacillin, methicillin, nafcillin, oxacillin, axlocillin, carbenicillin, mezlocillin, piperacillin, ticarcillin, cefadroxil, cefazolin, cephalixin, cephalothin, cephapirin, cephradine, cefaclor, cefacmandole, cefmetazole, cefonicid, ceforanide, cefotetan, cefoxitin, cefprozil, cefuroxime, loracarbef, cefixime, cefoperazone, cefotaxime, cefpodoxime, ceftazidime, ceftiofur, ceftizoxime, ceftriaxone, moxalactam, aztreonam, imipenem, eropenem, ciprofloxacin, enrofloxacin, difloxacin, orbifloxacin, marbofloxacin, chloramphenicol, thiamphenicol, florfenicol, chlortetracycline, tetracycline, oxytetracycline, doxycycline, minocycline, erythromycin, tylosin, tlimicosin, clarithromycin, azithromycin, lincomycin, clindamycin, gentamicin, amikacin, kanamycin, apramycin, tobramycin, neomycin, dihydrostreptomycin, paromomycin, sulfadmethoxine, sfulfamethazine, sulfaquinoxaline, sulfamerazine, sulfathiazole, sulfas alazine, sulfadiazine, sulfabromomethazine, suflaethoxypyridazine, vancomycin, teicoplanin, ramoplanin, decaplanin, rifampin, nitrofuran, virginiamycin, polymyxins, and tobramycin.

24. The method of arrangement 21, wherein the anti-fungal agent comprises one or more of itraconazole, ketoconazole, fluoconazole, voriconazole, griseofulvin and amphotericin B.

25. The method of any one of the preceding arrangements, wherein the infectious disease comprises a cold, pneumonia, or influenza.

26. The method of any one of the preceding arrangements, wherein the infectious disease comprises a viral infection, fungal infection, a bacterial infection, and/or a parasitic infection.

27. The method of any one of the preceding arrangements, wherein the infectious disease comprises an infection by one of more of a coronavirus, herpes simplex virus, papilloma virus, parainfluenza virus, influenza virus, hepatitis virus, Coxsackie Virus, herpes zoster virus, measles virus, mumps virus, rubella virus, rabies virus, hemorrhagic fever virus, human immunodeficiency virus (HIV), and H1N1 virus.

28. The method of any one of the preceding arrangements, wherein the infectious disease comprises a coronavirus infection.

29. The method of arrangement 28, wherein the coronavirus infection comprises an infection by a Middle East respiratory syndrome coronavirus (MERS-CoV), severe acute respiratory syndrome coronavirus (SARS-CoV), or SARS-CoV-2 (COVID-19).

30. The method of any one of the preceding arrangements, wherein the infectious disease comprises an infection by one of more of Escherichia coli, Salmonella enterica, Shigella dysenteriae, Vibrio cholerae, Vibrio vulnificus, Vibrio parahaemolyticus, Virio vulnificus, Campylobacter jejuni, Klebsiella, Enterobacter, Serratia, Proteus, Providencia, and Morganella, Bacillus anthracis, Bacillus cereus, Clostridium tetani, Clostrium botulinum, Clostridium perfringens, Clostridium difficile, Mycobacterium tuberculosis, Legionella pneumophilla, Vibrio cholera, Staphylococcus aureus (such as Methicillin-resistant Staphylococcus aureus (MRSA)), Staphylococcus epidermidis, Staphylococcus saprophyticus, Streptococcus pyogenes, Streptococcus agalactiae, Enterococcus faecalis, Streptococcus bovis, Streptococcus pneumoniae, Streptococcus viridans, Pseudomonas aeruginosa, Corynebacterium diphtheriae, Listeria monocytogenes, Burcella, Francisella tularensis, Yersinia enterocolitica, Yersinia pseudotuberculosis, Yersinia pestis, Pasteurella multocida, Mycobacterium tuberculosis, Mycobacterium bovis, Mycobacterium avium, Mycobacterium leprae, Actinomyces israelii, Nocardia asteroides, Mycoplasma pneumoniae, Treponema pallidum, Borrelia brugdorferi, Leptospira interrogans, Chlamydia psittaci, Chlamydia trachomatis, Chlamydia pneumoniae, R. rickettsii, Coxiella burnetii, R. prowazekii, Gardnerella vaginalis, Lactobacillus, Peptococcus, Peptostreptococcus, Propionibacterium, Tropheryma, Burkholderia pseudomallei, and Burkholderia mallei.

31. The method of any one of the preceding arrangements, wherein the infectious disease is associated with acute respiratory distress syndrome (ARDS) and/or a cytokine storm.

32. The method of any one of the preceding arrangements, wherein a targeted treatment for the infectious disease is not available.

33. The method of any one of the preceding arrangements, wherein a vaccine effective for the infectious disease is not available.

34. The method of any one of the preceding arrangements, wherein the subject has acute respiratory distress syndrome (ARDS).

35. The method of any one of the preceding arrangements, wherein the subject has one or more comorbidities.

36. The method of arrangement 35, wherein the one or more comorbidities comprise hypertension, diabetes and coronary heart disease.

37. The method of any one of arrangements 1 to 34, wherein the subject does not have a comorbidity.

The above description discloses several methods and materials. This disclosed matter is susceptible to modifications in the methods and materials, as well as alterations in the fabrication methods and equipment. Such modifications will become apparent to those skilled in the art from a consideration of this disclosure or practice of the subject matter disclosed herein. Consequently, the specific embodiments disclosed herein are not intended to be limiting, and all modifications and alternatives coming within the true scope and spirit of the present disclosure are intended to be covered.

What is claimed is:

1. A method of treating a subject having an infectious disease, comprising co-administering to a subject having an infectious disease:
a therapeutically effective amount of an ANS optimizing therapy; and
a treatment for the infectious disease,
wherein co-administering the ANS optimizing therapy improves the prognosis for treatment of the infectious disease.
